(19) United States
(12) Patent Application Publication
(10) Pub. No.: US 2011/0136741 A1
(43) Pub. Date: Jun. 9, 2011

(54) DICARBA-ANALOGUES OF OCTREOTIDE

(76) Inventors: Mauro Ginanneschi, Prato (IT); Debora D'addona, Poggibonsi (IT); Alessandra Di Cianni, Pistoia (IT)

(21) Appl. No.: 13/003,563

(22) PCT Filed: Jul. 8, 2009
(86) PCT No.: PCT/IB09/52965

§ 371 Date: Jan. 10, 2011

(30) Foreign Application Priority Data
Jul. 8, 2008 (IT) FI2008A000123

(51) Int. Cl. A61K 38/12; C07K 7/64; A61P 35/00; A61P 19/02

(52) U.S. Cl. 514/16.6; 530/317; 514/21.1; 514/19.2

(57) ABSTRACT

Analogues of octreotide, their preparation and use are described.

### FIELD OF THE INVENTION

The invention relates to analogue derivatives of octreotide.

### STATE OF THE ART

Somatostatin-14 (SRIF-14) is a cyclotetradecapeptide containing a disulphide bridge between two cysteine residues (Formula 1);

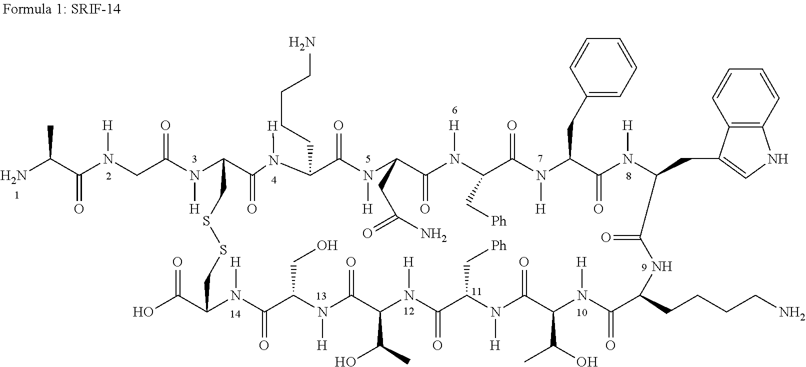

SRIF-14 is an important regulator of endocrine and exocrine secretion and inhibits the release of various other hormones such as glucagon, growth hormone (GH), insulin, gastrin and others.

Moreover, in view of its distribution in various regions of the brain and spinal cord, somatostatin is thought to play a role in neuronal transmission.

On the basis of their structural, phylogenetic and pharmacological characteristics, the SRIF receptors can be divided into two main classes: SRIF₁ which comprises receptors sst2, sst3, sst5, and SRI F₂ which comprises receptors sst1 and sst4. The clinical use of somatostatin, however, has been limited by a number of disadvantages such as its short half-life in blood (<3 minutes).

To overcome these disadvantages a large number of agonists have been designed and synthesized with the expectation of their possible clinical application in treating diseases such as: hypersecretory tumours, acromegaly, diabetes, rheumatoid arthritis, gastrointestinal disorders and disorders of the central nervous system, as published in the vast amount of literature.

To date numerous and potent somatostatin analogues have been discovered, normally formed of cyclic peptides of 6-11 amino acids connected together by disulphide bridges or backbone bonds.

However, despite the large number of synthesized products, only two or three are currently used in clinical practice, including in particular sandostatin® or octreotide® (SMS 201-995) which has demonstrated a very high affinity for subtype sst2 receptors and has assumed particular importance mainly as a radiolabelled drug in the diagnosis and therapy of certain tumours.

Octreotide is a cyclic octapeptide containing the pharmacophore motif Phe-D-Trp-Lys-Thr which is considered to be the active part due to the type-II' β-turn which involves D-Trp-Lys residues (Formula 2).

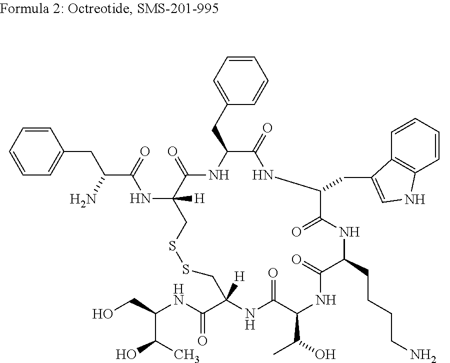

It is interesting to note how octreotide is very active towards the hsst2 and hsst5 receptors and has an in vivo half-life of 30 to 90 minutes.

The molecule is used in particular as a carrier for radioisotopes (⁹⁰Y-DOTA-D-(Phe¹, Tyr³)-octreotide (SMT487 or OctreoTher), for radiotherapy and ¹¹¹In-DTPA-D-Phe¹, Tyr³)-octreotide (Octreoscan) for diagnostics. On the other hand, the —S—S— group contained in octreotide is sensitive to endogenous and exogenous reducing and basic agents, but can primarily be opened during the labelling with radioisotopes such as ^{99m}Tc and ¹⁸⁸Re which takes place in a reducing environment [Fichna J, Janecka A. Synthesis of target-specific radiolabeled peptides for diagnostic imaging. Bioconjugate Chem. 2003; 14: 3-17].

In European patent EP 1,598,366 derivatives of octreotide are described whereby disulphide bridges were substituted by dicarba bridges which, when subjected to binding tests, showed an unexpected affinity and selectivity for receptor subtype sst5 [D. D′Addona, A. Carotenuto, E. Novellino, V. Piccand, J. C. Reubi, A. Di Cianni, F. Gori, A. M. Papini, M. Ginanneschi, “Novel sst5-Selective Somatostatin Dicarba-Analogues: Synthesis and Conformation-Affinity Relationships,” J. Med. Chem., 2008, 51(3), 512-520].

In the light of the aforesaid, it is of evident importance to provide new octreotide analogues which have not only a greater efficiency and stability in vivo but are also able, after conjugation to suitable chelating agents, to withstand a labelling with isotopes that takes place under reducing conditions such as to induce opening of the octreotide disulphide bridge. As even small variations in the peptide sequence and ring size can have considerable consequences on receptor affinity and selectivity, we have prepared a series of new dicarba-analogues of sandostatin to extend the range of their capacity for binding to the different sstrs. Moreover, it has been surprisingly proven that by changing the stereochemistry of one or two of the first amino acids, it is possible to transform agonist activity into antagonist activity, with certain advantages for the use of these analogues as radiopharmaceuticals. The final aim is to obtain robust analogues which have affinity and possible selectivity for several receptor subtypes, with agonist activity if they are required to be used directly as cell growth inhibitors or even as radiopharmaceuticals. However, for applications as radiopharmaceuticals only, whether in the diagnostic or therapeutic field, the antagonistic activity is more suitable.

The most common amino acid abbreviations reported below are those recommended by the IUPAC-IUB Joint Commission (Eur. J. Biochem, 1984, 138, 9-37). The other abbreviations are either explained or can be found in publications of the art.

Numbering of the amino acids in formulas (I) and (II) below refers to their position in the formula of native SRIF-14.

### DETAILED DESCRIPTION OF THE INVENTION

The present invention enables the aforesaid problem to be overcome by way of the octreotide analogues of general formula (I)

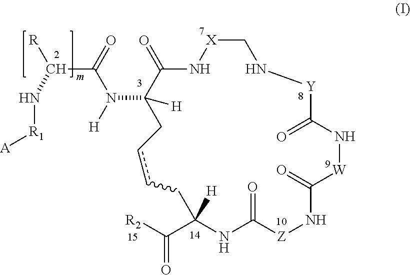

in which:

the symbol [symbol] is a double or single bond;

the symbol [symbol] indicates that the configuration of the chiral carbon atoms in positions 2 and 3 can be D or L (R or S);

the symbol [symbol] indicates that the configuration of the double bond can be E or Z;

Λ=H or a chelating group able to bind a radioisotope;

R is H or a phenyl or a benzyl or a p-OR′ substituted benzyl residue in which R′ is

H, or a linear or branched (C₁-C₆) alkyl substituent or benzyl; otherwise R is p-F-benzyl or p-Cl-benzyl (amino acid Cpa²);

if R=H, m varies from 1 to 6; if R is other than H, then m=1.

R₁ is a bond or a residue of an amino acid chosen from Gly, Asp or β-alanine;

if R₁ is an amino acid residue, then R is other than H, m=1 and Λ is bound to the terminal NR, group;

R₂ is —OH or —NH₂ or —NH—Q where Q is a cycloalkyl residue or an amino acid residue chosen from: Thr(ol), Thr, Thr-NH₂, Asp, Asn, Glu, Gln, Phe, Tyr, Tyr-NH₂, Lys, Orn, Nal-NH₂; in the definition of substituent Q, the term cycloalkyl means for example cyclopentyl or cyclohexyl;

X is an amino acid residue chosen from: D- or L-Phg, or D- or L-Tyr, or D- or L-Phe either unsubstituted or ortho- or para- substituted with the OR″ group in which, if the group is in the ortho- position, R″ is H or a linear or branched (C₁-C₆) alkyl or benzyl; if the substituent group is in the para- position, then R″ cannot be H but one of the aforedefined substituents; otherwise X is 1-Nal or 2-Nal as such or substituted on the A or B ring by a OR″ group in which R″ is H, a linear or branched (C₁-C₆) alkyl, or a benzyl.

Y is D-Trp, or D-Trp in which the NH of the indole ring has become NR′″ where R′″ is —CONH₂ or —COCH₃; or Y is D-Trp substituted on the benzene ring in position 7 with R″″, where R″″ is —OCONH₂, or —NHCONH₂ or —NHCOCH₃; otherwise Y is D-Aph-Cbm (4-aminophenylalanine-N-carbamoyl) or D-Agl [(N^{β}Me,2-naphthoyl) amino glycine] or D-2-Nal, or D-2-Nal substituted on the A or B ring by a OR″ group in which R″ is H, a linear or branched (C₁-C₆) alkyl, or benzyl;

W is Lys or IAmp [4-(N-isopropyl)-aminomethylphenylalanine] or 3-(N-isopropyl) amino-Tyr; or W is Har (homoarginine), or Hci (homocitrulline);

Z is an amino acid residue chosen from: homo-Thr, Thr(ol), Thr(Ac), Ser, Ser(Ac), homo-Ser, 4-hydroxy-Val, D- or L-Phe ortho- or para-substituted with a —OR′″″ group where, if X is other than L-Phe, R′″″ can be H or a linear or branched alkyl residue or a benzyl residue or a substituted benzyl or 1- or 2-naphthylmethyl; if X=L-Phe, then R′″″ is H or 1- or 2-naphthylmethyl; otherwise Z is 1- or 2-Nal, either as such or substituted on the A or B ring with a —OCH₃ or —OBz group.

Preferably the term “chelating group able to bind to a radioisotope” means a known chelating agent of macrocyclic nature, specially adapted to coordinate ions such as ¹¹¹In and ⁹⁰Y, as reported for example in the aforesaid EP 1,598,366; or a molecule able to coordinate ^{99m}Tc or ¹⁸⁸Re such as tricarbonyl or N-[N-(3-diphenylphosphinoproprionyl)-glycyl]-cysteine (PN₂S), this latter being described in Inorg. Chem. 2003, 42, 950-959; all these chelating agents are well-known to all those who work in radiodiagnostics.

The compounds of formula (I) of the invention are prepared using the normal synthesis techniques starting from known or easily prepared products.

In particular, according to the invention the products of formula (I), when R₂ =—NH—Q and Q is an amino acid, can be prepared by a process comprising the following steps:

a) the linear heptapeptides (i.e. devoid of the N-terminus amino acid) are prepared by SPPS according to known methods by anchoring the sequence to a derivatized chlorotrityl resin, often already supplied with the first amino acid (C-terminus) (for example, if this is Thr-2-ol, the resin is H-L-Thr(t-Bu)-ol-2-chlorotrityl, produced by Iris Biotech, Marktredwitz, Germany). If R₂=—NH—Q and Q is an amino acid with a terminal CONH₂ group, the support is 4-(2′,4′-dimethoxyphenyl-Fmoc-aminomethyl)-phenoxymethyl polystyrene resin, known as Rink amide resin or, may be, Rink amide-acetamido-norleucylaminomethyl resin, known as Rink amide AM resin. The same takes place if R₂=NH₂;

b) the peptides linked to the resin are cyclized by means of second generation Grubbs catalysts or first generation Hoveyda-Grubbs catalysts.;

c) the amino acid in position 2 is then added;

d) the cyclopeptides are deprotected and separated from the resin;

e) the cyclopeptides are purified by RP-HPLC; or, after step (e),

e′) the cyclopeptides are reduced in solution with H₂ and a Pd catalyst and purified as aforesaid;

or, when Z is an amino acid containing a substituent removable with a H₂/Pd catalyst, then after step c):

d′) the cyclopeptides on the resin are reduced with a Wilkinson catalyst and then separated from the resin and purified as aforesaid; or

the cyclopeptides, either unsaturated or hydrogenated, anchored to the resin can be further conjugated to an amino acid as aforedefined and/or to a macrocyclic chelating agent at the NH₂-terminus amino acid, by following the process previously described in said European patent; or

a PN₂S group is constructed on the unsaturated or hydrogenated cyclopeptides linked to the resin, as illustrated in the following example, after which the entire molecule is separated from the resin and purified by HPLC.

According to a particular embodiment of the invention, reference is made to compounds of formula (II) which are designed exclusively for their antagonist activity:

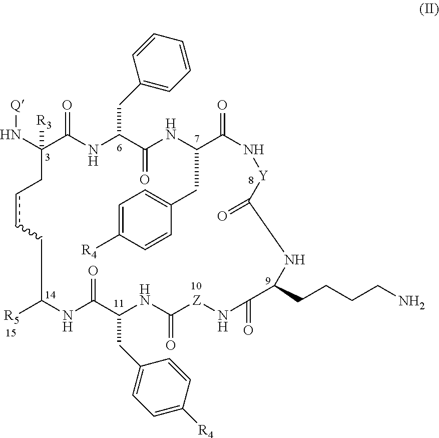

in which:

the symbol [symbol] is a double or single bond;

the symbol [symbol] indicates that the configuration of the relative chiral carbon atom can be D or L (R or S);

the symbol [symbol] indicates that the configuration of the double bond can be E or Z;

Q′=−Λ, —CONH-Λ, —COCH₂NH-Λ, —(CH₂)ₙ—NH-Λ, in which n is between 1 and 6, or an amino acid residue chosen from Gly or from L- or D- amino acids such as Phe, Tyr, Cpa, Dab, Lys, Hly (homolysine), Orn, Arg, Har (homoarginine), in which the terminal amino group is —NH-Λ;

−Λ is H or a chelating group as aforesaid;

R₃=H or —(CH₂)ₘ-K where m is between 0 and 8 and K is —NH₂; or a guanidine or imidazole residue;

R₄ is H or —OH or —OCH₃ or —OBz;

Y is D-Trp or substituted D-Trp in which NH of the indole ring has become NR′″ where R′″ is —CONH₂ or —COCH₃; or Y is D-Trp substituted on the benzene ring in position 7 with R″″, where R″″, is —OCONH₂, or —NHCONH₂ or —NHCOCH₃;

otherwise Y is D-Aph-Cbm (4-aminophenylalanine-N-carbamoyl) or D-Agl [(N^{β}Me,2-naphthoyl) amino glycine] or D-2-Nal, or D-2-Nal substituted on the A or B ring by a OR″ group in which R″ is H, linear or branched (C₁-C₆) alkyl, or benzyl;

Z is an amino acid residue chosen from Thr, Thr(Ac), Thr(ol), homo-Thr, Ser, Ser(Ac), homo-Ser, Hyl (δ-hydroxy-lysine), Tyr, or Tyr(OR″″″) where R″″″ is a linear or branched C₁₋₆ alkyl group, or a benzyl or a substituted benzyl with hydrophobic groups ; or R″″″ is 1- or 2-naphthylmethyl; otherwise Z is 1- or 2-Nal, either as such or substituted on the A or B ring with a —OCH₃ or —O-Bz group. R₅=—NH₂, —CONH₂, —COOH; —CONHR₆ where R₆=C₂-C₆ alkyl or cycloalkyl, an aromatic or heteroaromatic ring or an amino acid residue chosen from: Asp, Asn, Glu, Gln, Thr(ol). In the definition of the substituent R₆, the term cycloalkyl means for example cyclopentyl or cyclohexyl, whereas examples of an aromatic and heteroaromatic ring are respectively: phenyl and hydroxyphenyl, pyrrole and imidazole.

The compounds of formula (II) of the invention described hereinafter are prepared using known methods and known reagents or those easily obtainable by known methods.

In particular when Q′=—H, R₃=H, R₄=H, R₅=—COOH or —CONH₂ the following is carried out:

a) linear octapeptides are prepared by SPPS using known methods by anchoring the sequence to a suitable solid support [for example if R₅=—COOH, the resin is a resin containing a p-benzyloxybenzyl alcohol functional group (Wang resin); if R₅=—CONH₂, the first amino acid is anchored to the Rink amide or the Rink amide AM resin];

b) the linear peptides on the resin are cyclized by means of second generation Grubbs catalysts or first generation Hoveyda-Grubbs catalysts;

c) the cyclopeptides are deprotected and detached from the resin;

d) the cyclopeptides are purified by RP-HPLC;

or, if R₅ is —CONHR₆, the amino acid residue R₆ is previously bound to a suitable resin (such as a Wang resin) or a Rink Amide resin as described in step (a).

Subsequent steps follow on as aforedescribed:

The processes for preparing these products are fully illustrated in the following examples.

### Example 1

Synthesis of the linear heptapeptide Fmoc-Hag³(allylglycine)-1-Nal⁷-D-Trp⁸-Lys⁹-Thr¹⁰ -Haq¹⁴-Thr(ol)¹⁵

The peptide was prepared in a Teflon reactor equipped with a porous polystyrene septum, using the Fmoc-SPPS strategy on pre-swollen H-L-Thr(tBu)-ol-2-chlorotrityl resin.

The coupling steps were carried out by adding two equivalents of protected amino acids, activated by HATU, and four equivalents of NMM in DMF, then agitating for 45 minutes for each coupling, and monitoring by a qualitative test with ninhydrin (Kaiser test).

On completion of the linear peptide synthesis, a micro-cleavage was carried out on 5 mg of resin. The resin was treated with TFA/H₂O/1,2-Ethanedithiol (EDT)/phenol (94:2:2:2; 3 h) and filtered, the solution was concentrated under reduced pressure, the peptide was precipitated with Et₂O, centrifuged, dissolved in water and lyophilized. Analysis by RP-HPLC of the Fmoc-protected crude peptide [Phenomenex Jupiter C18 column (5 μm, 250×4.6 mm) with: 1 mL/min flow rate; eluent: A (0.1% TFA in H₂O) and B (0.1% TFA in CH₃CN)] has shown the presence of the linear peptide with about 95% purity, without traces of isomers due to racemisation of the amino acids.

ESI-MS: [M+H]⁺ 1134.9; calculated [M]⁺1133.56.

### Example 2

Synthesis of the cyclic unsaturated peptide of formula (I) in which -ΛA is H, R=benzyl, R₁ is a bond, CH² is in D configuration, CH³ is in L configuration, X=1-Nal, Y=D-Trp, W=Lys, Z=Thr, R₂=-NHThr(ol)

The on-resin heptapeptide was re-swollen for 2 hours in anhydrous DCM. The reactor was heated to 45° C. and there was added a solution of second generation Grubbs catalyst, [(L)(L′)X₂Ru=CHR where L is a phosphine ligand, L′ is a heterocyclic carbene (1,3-dimesityl-imidazol-2-ylidene), X is a Cl atom and R is a phenyl group] in DCM (0.5 mol equiv.) and the suspension agitated for 24 hours. The resin was washed with DCM, DMF and MeOH, then swollen with DMF. Fmoc-Hag was deprotected with 20% piperidine, and the cyclic peptide coupled to Fmoc-D-Phe as aforedescribed. The cyclic peptide was deprotected and detached from the resin as aforedescribed. The solution was concentrated with Et₂O to provide the crude dicarba-analogue which was suspended in water, the precipitate being eliminated by centrifugation. The compound was purified by semi-preparative RP-HPLC [Jupiter C18 column (10 μm, 250×10 mm) using the same eluent system as aforesaid (20% -60% of B in 20 minutes), 4 mL/min. HPLC purity>98% (yield: 11%)

ESI-MS: found [M+H]⁺ 1032.0, [M+2H]²⁺ 517.2, [M+Na]⁺ 1054.25; calc. [M+H]⁺ 1030.53

### Example 3

Synthesis of the unsaturated cyclic peptide of formula (I) in which −Λ is the chelating group DOTA, R=benzyl, R₁ is a bond, CH² is in D configuration, CH³ is in L configuration, X=1-Nal Y=D-Tr W=Lys, Z=Thr, R₂=-NHThr (ol)

Fmoc-D-Phe² deprotection of the peptide of formula I bound to the resin, as in example 2, was carried out with 20% piperidine; subsequently 2 mol equiv. of DOTA-tri-(t-Bu) ester in DMF, 2 mol equiv. of HATU in DMF and then 4 mol equiv. of NMM were added to the resin. The equivalents were calculated based on a resin substitution level of 0.5 mmol/g. After 45 minutes the resin was washed and the DOTA-conjugated peptide was cleaved from the resin as aforedescribed. The tri-(t-Bu) DOTA-protective groups were cleaved along with the peptide. The crude peptide was dissolved in water and lyophilized, then purified by RP-HPLC (20%-60% of B in 20 minutes; yield: 29%)

ESI-MS: found [M+H]⁺ 1417.72, [M+2H]²⁺ 709.79; calc. [M+H]⁺ 1416.71.

### Example 4

Synthesis of the unsaturated cyclopeptide of formula (I) where −Λ is the chelating group PN₂S, R=phenyl, R₁ is a bond CH² is in D configuration, CH³ is in L configuration, X=1-Nal, Y=D-Trp, W=Lys, Z=Thr, R₂=-NHThr (ol)

The PN₂S group is able to chelate ^{99m}Tc and ^{186/188}Re and, at the same time, to utilize the carboxyl group of the cysteine to bind to the carrier peptide. Based on our experience with SPPS, we decided not to couple the preformed pseudo-tripeptide PN₂S directly to the dicarba-analogue of formula (I) but to construct it step by step on the cyclopeptide anchored to the resin.

After deprotecting the unsaturated cyclopeptide of formula (I) with 20% piperidine, the dicarba-analogue was coupled with Cys(Trt) then with Gly and finally with the succinimide ester of 3-diphenylphosphino propanoic acid. As the thiol and phosphino groups are easily oxidizable, the detachment procedure from resin was carried out under argon and the cleavage mixture was degassed. We thus obtained the dicarba-analogue conjugated to the chelating group PN₂S with a total yield of 4%. Purification of the crude product (PR-HPLC) was successfully achieved in degassed solvents, the collected fractions being rapidly frozen and lyophilized to avoid degradation of the oxydizable groups. Eluent: 40% -70% of B in 20 minutes.

ESI-MS: found [M+H]⁺ 1432.41, [M+2H]²⁺ 717.0; calcd. [M+H]⁺ 1430.63.

### Example 5

Reduction of the alkyl chain on the peptide ring of the analogue of formula (I) (reduction of the —CH═CH—group)

The pure unsaturated cyclooctapeptide of formula (I) cited in example 2, in which −Λ is H, was dissolved in anhydrous MeOH and 10% (w/w) of 20% Pd(OH)₂/C was added. The reaction vessel was purged with a N₂ stream and then subjected to a H₂ flow. The suspension was agitated at 30° C. for 24 h, filtered over Celite, the solvent evaporated under reduced pressure and the crude product was lyophilized. The product was purified with semi-preparative RP-HPLC (from 20% to 60% of B in 20 minutes) to provide the pure cyclopeptide with —CH₂-CH₂-alkyl closure (reduction yield: 47%).

ESI-MS: found [M+H]⁺ 1034.0, [M+2H]²⁺ 517.9, [M+Na]⁺ 1055.23; calc. [M+H]+ 1032.15.

### Example 6

Synthesis of the linear octapeptide Fmoc-Hag³-Phe⁶-Phe⁷-D-Trp⁸-Lys⁹-Thr¹⁰-Phe¹¹-Hag¹⁴-NH₂

To obtain the C-terminus amide bond, both the aforementioned Rink amide and Rink amide AM resins were used. The process is the same as described above for the linear heptapeptide. On conclusion of chain lengthening, the linear peptide was detached from the resin as aforedescribed. The Fmoc-protected peptide was analyzed by RP-HPLC and showed a purity of around 95%.

ESI-MS: found [M+H]⁺ 1291.3; calc. [M+H]⁺ 1289.63.

### Example 7

Sythesis of the unsaturated cyclic peptide of formula (II) where Q=H, R₃=H, R₄=H, Y=D-Trp, R₅=—CONH₂.

The RCM reaction was applied directly to the linear octapeptide bound to the Rink Amide AM resin by means of the same second generation Grubbs catalyst used to form the cyclic analogue of formula (I) reported in example 2, using the same conditions. Cleavage was carried out as aforedescribed and the crude product was purified by RP-HPLC (30% -60% of B in 20 min) to give the pure compound (total yield: 10%).

ESI-MS: found [M+H]+ 1041.1; calc. [M+H]+ 1039.53.

### Example 8

Reduction of the alkyl chain on the peptide ring of the analogue of formula (II) (reduction of the —CH═CH—group)

The pure unsaturated cyclooctapeptide of formula II, as in example 7, in which Q is H, was dissolved in anhydrous MeOH followed by addition of 10% (w/w) of 20% Pd(OH)₂/C. The experimental details are as described in example 5. Analytical RP-HPLC showed a purity of around 70%. Purification was carried out by RP-HPLC (20% -60% of B in 20 minutes).

ESI-MS: found [M+H]+ 1043.1; calc. [M+H]+ 1041.54 The products thus obtained, if −Λ is H and if they are agonists, can be used for the preparation of pharmaceutical compositions according to the normal methods known in the pharmacopeia for the use of equivalent active principles. Said pharmaceutical formulations can be used for the direct treatment, for example of tumours, acromegaly, rheumatoid arthritis. Alternatively the products of formula (I) and (II), if −Λ is a macrocycle (such as DOTA) or a chelating agent such as PN₂S, can be radio-labelled with metals such as Fe-52, Mn-52m, Co-55, Cu-64, Ga-67, Ga-68, Tc-99m, In-111, I-123, I-125, I-131, P-32, Sc-47, Cu-67, Y-90, Pd-109, Ag-111, Pm-149, Re-186, Re-188, At-211, Bi-212, Bi-213, Rh-105, Sm-153, Lu-177, Au-198 and used for radiodiagnosis or radiotherapy. The compounds described in the preceding examples were tested in the first instance on cell cultures and showed considerable, and even selective, affinities for subtypes sstl, sst2, sst3 and sst5 in particular.

1-7. (canceled)

8. A compound of formula (I) 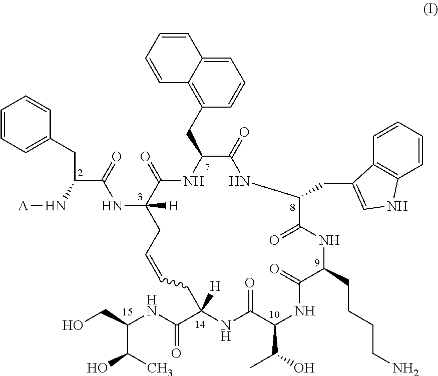
wherein:
the carbon atom in position 2 is a chiral carbon atom having a D configuration, the carbon atom in position 3 is a chiral carbon atom having an L configuration, [symbol] indicates a double bond E or Z configuration, and
Λ is H or DOTA.

9. A process for preparing the compound of claim 8 wherein Λ is H, the process comprising:
a) preparing a linear heptapeptide corresponding to the compound of formula (I), the preparing performed by solid phase peptide synthesis (SPPS), the solid phase peptide synthesis performed by anchoring the heptapeptide to a suitably derivatized chlorotrityl resin to obtain a heptapeptide linked to the resin;
b) cyclizing the heptapeptide linked to the resin by means of a second generation Grubbs catalyst to obtain a cyclopeptide linked to the resin;
c) adding an amino acid in position 2 of the cyclopeptide linked to the resin;
d) deprotecting and separating the cyclopeptide from the resin; and
e) purifying the cyclopeptide by Reverse Phase-High Performance Liquid chromatography (RP-HPLC).

10. A process for preparing the compound of claim 8 wherein Λ is DOTA, the process comprising:
a) preparing a linear heptapeptide corresponding to the compound of formula (I), the preparing performed by solid phase peptide synthesis (SPPS), the solid phase peptide synthesis performed by anchoring the heptapeptide to a suitably derivatized chlorotrityl resin to obtain a heptapeptide linked to the resin;
b) cyclizing the heptapeptide linked to the resin by means of second generation Grubbs catalysts to obtain a cyclopeptide linked to the resin;
c1) adding an amino acid in position 2 of the cyclopeptide linked to the resin;
c2) conjugating cyclopeptide linked to the resin to DOTA at the NH₂-terminus of the amino acid in position 2 of the cyclopeptide linked to the resin;
d) deprotecting and separating the cyclopeptide linked to the resin from the resin; and
e) purifying the cyclopeptide by Reverse Phase-High Performance Liquid chromatography (RP-HPLC).

11. A pharmaceutical composition comprising the compound of claim 8 as an active component.

12. The pharmaceutical composition according to claim 11, wherein the active component is comprised in an effective amount to treat one or more tumors.

13. The pharmaceutical composition according to claim 11, wherein the active component is comprised in an effective amount to treat acromegaly.

14. The pharmaceutical composition according to claim 11, wherein the active component is comprised in an effective amount to treat rheumatoid arthritis.

15. A radiopharmaceutical suitable for diagnosis and therapy of tumors, the radiopharmaceutical comprising the compound of claim 8, in which A is DOTA.
